(19) United States
(12) Patent Application Publication
(10) Pub. No.: US 2005/0054990 A1
(43) Pub. Date: Mar. 10, 2005

(54) Split-tip catheter divider

(76) Inventors: Joanna Graft, West Jordan, UT (US); Kenneth Berry, South Haven, MI (US); Huong Le, West Valley City, UT (US)

Correspondence Address: MORRISON & FOERSTER, LLP, 555 WEST FIFTH STREET, SUITE 3500, LOS ANGELES, CA, 90013-1024, US

(21) Appl. No.: 10/657,354

(22) Filed: Sep. 8, 2003

(51) Int. Cl. A61M 25/00

(52) U.S. Cl. 604/284; 604/43

(57) ABSTRACT

A dividing element for a split-tip catheter. The dividing element has a body that may contain openings for disposition on a tip section of a split-tip catheter. The openings are spaced apart by a distance sufficient to ensure separation between tip sections when the body is positioned thereon and may be configured to provide a tension to prevent relative movement of the tip section(s) with respect to the dividing element. The dividing element may be configured for attachment to a packaging tray and/or may contain weakened sections to facilitate detachment from the catheter.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A COMPACT DISK APPENDIX

Not applicable.

### BACKGROUND OF THE INVENTION

A primary goal of hemodialysis access is to provide a reliable and effective means of dialysis, which means that a sufficient volume of blood over a period of time must be removed from and returned to the patient. Because the contaminated and cleansed blood must be kept separate for an effective dialysis procedure, a dual lumen catheter is generally used. Dual lumen catheters are usually configured so that there is a shorter lumen that aspirates blood from a blood vessel of a patient to a dialysis machine where it is processed for the removal of toxins, and a longer lumen that infuses the purified blood to the patient. The shorter lumen utilized for aspiration is generally referred to as the “arterial lumen,” while the longer lumen utilized for infusion is generally referred to as the “venous lumen.” The reason for the different lengths is to minimize co-mingling of aspirated and infused blood.

The primary problems occurring in dual lumen dialysis catheters are blood clotting (thrombosis) and fibrin (the protein formed during normal blood clotting that is the essence of the clot) sheath formation. Thrombus and fibrin sheath formation can occlude distal tips of the dialysis catheter lumens, resulting in loss of catheter function when such an occlusion prevents blood flow. This typically occurs initially in the arterial lumen used for aspiration of blood from a patient. A secondary problem relates to the arterial lumen “sucking” against the vessel wall, in which the arterial lumen openings become fully occluded by the patient's vasculature.

To specifically address these problems, a new type of dialysis access catheter has been designed that utilizes independent “free floating” distal tip sections that separate at a distal end of the catheter to theoretically reduce the likelihood of potential occlusion and “sucking” during dialysis treatment. U.S. Pat. No. 6,001,079 to Pourchez and U.S. Pat. Nos. 5,947,953 and 6,190,349 to Ash et al., all incorporated by reference herein, are directed to said new type of catheter, hereinafter referred to as a “split-tip catheter.” Improvements on split-tip catheters are provided in co-pending application Ser. No. 10/371,774, filed Feb. 21, 2003, which is incorporated by reference herein and is subject to common assignment.

Due to the particular configuration of split-tip catheters (i.e., tip sections are separated along their length distal a dividing point) and their typical polyurethane make-up, a potential problem can occur in that the tip sections fuse or “knit” together over time. Such fusion is undesirable as the subsequent separation of the tip sections may be uneven or may render the catheter unusable for its intended purpose. While this potential problem can certainly present itself during the storage of the split-tip catheters, it may also occur during sterilization, packaging and/or shipping of the catheters. Moreover, this potential problem can present itself in split-tip catheters made of materials other than polyurethane. Chemical type solutions to the stated problem (for example, adding a thin coating to one or more adjacent surfaces on the tip sections) were ultimately proven to be unfeasible due to either insufficient prevention of fusing or the requirement of amounts so great that an undesirable excess resulted.

Thus, there is a need for a solution to maintain the separation between the tip sections of a split-tip catheter, following the manufacture thereof, to prevent possible fusion during sterilization, packaging, shipping and/or storage.

### BRIEF SUMMARY OF THE INVENTION

Accordingly, a primary object of the present invention is to provide a solution to maintain separation of tip sections of a split-tip catheter during various stages, including but not limited to, sterilization, packaging, shipping and/or storage. It is another object of the present invention to provide a split-tip catheter having tip sections that are prevented from fusing together during various stages following the manufacture thereof. It is another object of the present invention to provide a dividing element that would remain between the tip sections, yet be easy to remove by an end user. It is still another object of the present invention to provide a dividing element with a simple design. It is yet another object of the present invention to provide a dividing element that can be easily manufactured. It is another object of the present invention to provide a dividing element that can be easily attached to the split-tip catheter. It is still another object of the present invention to provide a dividing element that can be secured to a packaging tray. Various other objectives and advantages of the present invention will become apparent to those skilled in the art as more detailed description is set forth below.

As used herein, the following terms have the following meanings:

“Split-tip catheter” refers to a catheter having a body enclosing at least two lumens and a dividing point that separates at least two tip sections from one another distal thereto, each of the tip sections enclosing at least one lumen and being separated or separable from one another along their length.

“Dividing point” refers to a point along the length of the split-tip catheter distal to which at least two tip sections are separated or are separable from one another.

“Tip section” refers to a portion of the split-tip catheter, enclosing at least one lumen, which is separable or is separated from another tip section along its length distal to a dividing point.

“Inner surface” refers to a surface on a tip section that is facing or adjacent a companion tip section (a tip section that separates or is separable distal to the same dividing point).

In one embodiment of the present invention, the dividing element comprises a body with a first and second opening that are separated by a length long enough to prevent contact between the two tip sections when the body is positioned therebetween. The openings can comprise slits and can be closer to a proximal end than a distal end so that the distance between the second opening and the distal end enables the body to be attached to a packaging tray, which in turn enables removal of the catheter from the packaging tray without removal of the dividing element from the packaging tray. The body may be relatively thin to facilitate packaging. The body may be positioned on the catheter by inserting the venous tip section through the two openings, the body thereby separating the venous tip section from the arterial tip section.

In another embodiment of the present invention, the dividing element may include weakened sections such that the dividing element can be removed from a catheter on which it is disposed by applying force to the dividing element in a direction away from the catheter, thereby splitting the weakened sections. Other embodiments of the present invention include a sheet of material with a strip of adhesive disposed thereon, where the sheet is rolled around one of the tip sections and adhered to itself, a piece of material wedged between adjacent tip sections such that the dividing element would remain in place until pulled out by the end user, and a thin-walled tube that could be fit over the shorter of adjacent tip sections, the lumen of the tube being fashioned to provide a friction fit.

These and other embodiments, features and advantages of the present invention will become more apparent to those skilled in the art when taken with reference to the following more detailed description of the invention in conjunction with the accompanying drawings that are first briefly described.

### BRIEF DESCRIPTION OF THE DRAWINGS

FIG. 1 is a side perspective view of a split-tip catheter.

FIG. 2 is a top view of a dividing element according to the present invention.

FIG. 3 is a close-up view of a distal end of the split-tip catheter of FIG. 1, with the dividing element of FIG. 2 disposed thereon.

FIG. 4 is a top view of a dividing element according to the present invention.

FIG. 5 is a top view of a dividing element according to the present invention.

### DETAILED DESCRIPTION OF THE INVENTION

The following detailed description should be read with reference to the drawings, in which like elements in different drawings are identically numbered. The drawings, which are not necessarily to scale, depict selected preferred embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

The present invention is directed to a dividing element for a split-tip catheter, which is used to keep separate the tip sections thereof during post-manufacturing handling, such as sterilization, packaging and shipping. Although a split-tip catheter could contain multiple tip sections and/or tip sections of equal lengths (and certainly such split-tip catheters are contemplated in connection with the present invention), it typically contains two tip sections with different lengths—a shorter (arterial) tip section and a longer (venous) tip section. Thus, unless otherwise noted, the specific embodiments discussed herein will be in reference to a typical split-tip catheter.

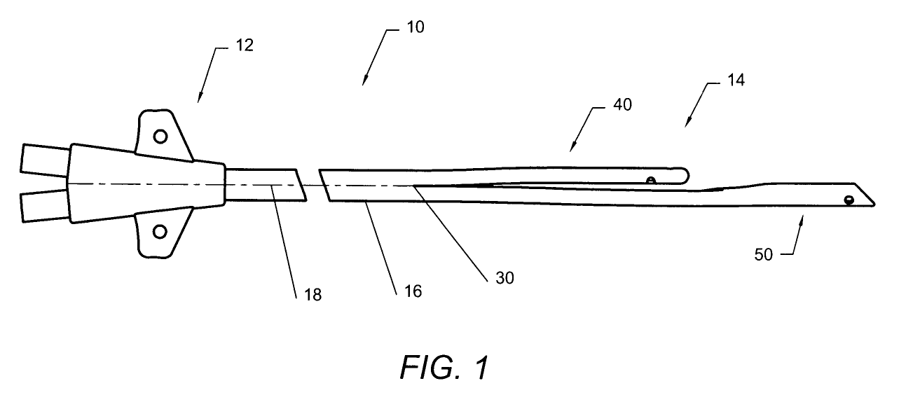

Referring to FIG. 1, a split-tip catheter 10 is shown having a proximal end 12 and a distal end 14. The proximal end of the catheter 12 is attached to a Y-connector, which in turn is connected to extension tubing. The catheter 10 has two lumens defined by an outer wall 16 and a bisecting planar septum 18. The two lumens extend from the proximal end of the catheter 12 to the distal end of the catheter 14 and have a D-shape configuration due to the planar septum 18. Of course, this is not the only possible configuration and certainly the lumens could assume a variety of shapes, including circular, crescent-shaped, oval, etc. The distal end 14 of the catheter 10 is separated into tip sections 40 and 50 distal to a dividing point 30. While only two tip sections are shown in FIG. 1, it is certainly possible for a plurality of tip sections to branch from the dividing point 30, depending, for example, on the number of lumens within the catheter 10. Thus, it is contemplated that three, four or more tip sections could separate or be separable distal to the dividing point 30, corresponding to three, four or more lumens within the catheter 10. In addition, although only one dividing point is illustrated in FIG. 1, it is contemplated that the split-tip catheter could contain two or more dividing points.

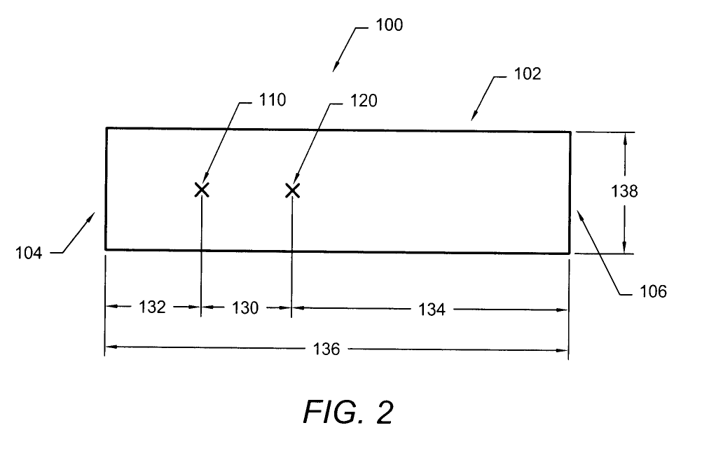

Referring to FIG. 2, a first embodiment of the divider element according to the present invention is illustrated. Divider element 100 includes a body 102, having a proximal end 104, a distal end 106, a length 136 and a width 138. Near the proximal end 104 of the body 102 is disposed a first opening 110 and a second opening 120, each in the form of an “X” shaped slit. First and second openings 110, 120 are spaced apart by a length 130, the first opening being spaced from the proximal end by a length 132 and the second opening being spaced from the distal end by a length 134. In one embodiment, the length 130 corresponds substantially to the length of the arterial tip section 40, depending on the size of the catheter, although ideally, the length 130 would be long enough to accommodate all catheter sizes. Thus, for example, the length 130 in one embodiment would be approximately 2 inches so that it would accommodate both 14.5 Fr and 16 Fr catheters, maintaining separation between the arterial and venous tip sections.

As shown in FIG. 2, the first opening 110 is positioned nearer to the proximal end 104 than is the second opening 120 to the distal end 106 (i.e., length 132 is less than length 134). This configuration has been found to be optimal in attaching the divider element 100 to a packaging tray (not shown), while facilitating overall appearance and functionality of the packaged kit. Specifically, the distal end 106 can be directly attached to the packaging tray after being positioned on the catheter so that the divider element 100 remains with the packaging tray when the catheter is removed. In one embodiment, the length 134 is approximately 3 times longer than length 132 (for example, length 134 is approximately 3 inches while length 132 is approximately 1 inch). Of course, other configurations of the openings are certainly possible, including openings that are spaced as a group closer to the distal end (i.e., length 132 is greater than length 134) and openings that are spaced the same distance from the proximal and distal end respectively (i.e., length 132 is equal to length 134).

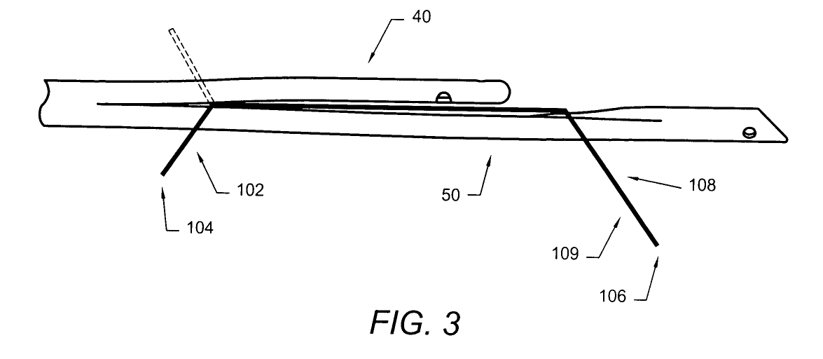

As illustrated, the first and second openings are in the form of “X” shaped slits, which is advantageous, for example, due to the gripping action of the slits onto the tip section which is placed therethrough and the ease of manufacture compared to other types of openings. It should be noted, however, that various shapes of openings are also possible and would be within the scope of the present invention. Referring to FIG. 3, the dividing element 100 is shown positioned on the catheter 10. In this embodiment, the venous tip section 50 is slid through the first opening 110 from a top surface 108 and through the second opening 120 from the opposing bottom surface 109. Shown in phantom is an alternate embodiment where arterial tip section 40 is slid through the first opening 110 from the bottom surface 109 and the venous tip section 50 is slid through the second opening 120 from the bottom surface 109. In each instance, body 102 is positioned between the arterial and venous tip sections 40, 50, with top surface 108 adjacent the arterial tip section 40 and the bottom surface 109 adjacent the venous tip section 50. As illustrated, the proximal end 104 overlap is minimal, while the distal end 106 overlap is more substantial, which furthers the goals of packaging as discussed above. It should be appreciated that the width 138 of the body 102 is equivalent to or greater than the width of at least the narrower of the tip sections (or both tip sections if they are the same width) it is separating so that contact between the two is prevented.

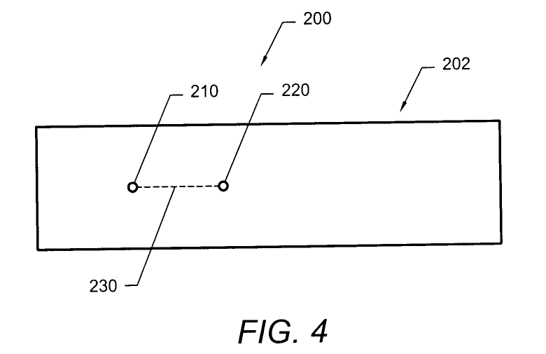

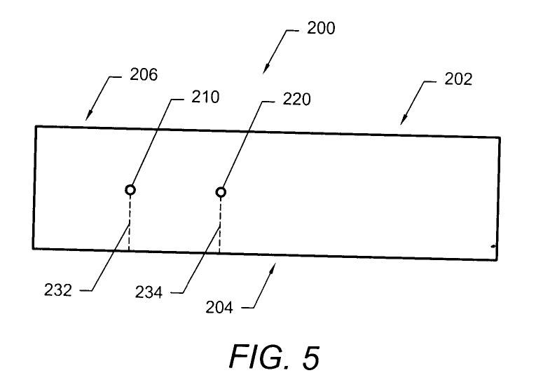

FIGS. 4 and 5 illustrate an alternate embodiment of the present invention, showing a dividing element 200, having a body 202 and first and second openings 210, 220, respectively. In FIG. 4, a weakened section 230 is positioned between the first and second openings 210, 220. Weakened section 230 can be in the form of a perforation or other alteration that is configured to maintain unity under normal handling and movement, but which will split or separate along its length when a force is applied in a certain direction. Dividing element 200 can be positioned onto a split-tip catheter similar to that shown in FIG. 3 (or in any other manner that would prevent contact between the arterial and venous tip sections). In the instance that dividing element 200 is positioned solely on the venous tip section (FIG. 3), it can be simply removed by grasping and pulling away from the catheter so that the body 202 splits along weakened section 230 and separates from the catheter.

In FIG. 5, a weakened section 232 is positioned between the first opening 210 and a side 204 of the body 202, and a weakened section 234 is positioned between the second opening 220 and the side 204. Removal involves grasping the body 202 at side 206, opposite of side 204, and pulling away from the catheter so that the body 202 splits along weakened sections 232, 234 and separates from the catheter. One of skill in the art will appreciate that while two specific embodiments of dividing element 200 have been illustrated, with particular positioning of a weakened section or sections, there are many other configurations involving the positioning of weakened section(s) that would equally be within the scope of the present invention.

Divider elements 100 and 200 may be made of polyethylene and, in particular, spun bond high density polyethylene (for example, Tyvek®), as well as many other suitable materials. Importantly, the material chosen should have attributes consistent with the objects of the present invention, including, but not limited to, resistant to bonding to the catheter material, light weight, inexpensive, durable, easy to handle and splittable. The divider elements 100 and 200 may also be very thin, for example, having a thickness of approximately 1-3 mm, provided that enough material is present to prevent fusion between the adjacent tip sections between which it is positioned, or may be thicker, having a thickness of approximately 10-15 mm. Of course, various thicknesses are possible, all of which would be within the scope of the present invention. Moreover, divider elements 100 and 200 may include more than two openings for use with split-tip catheters having more than one dividing point or more than two tip sections as explored above and in co-pending application Ser. No. 10/371,774, filed Feb. 21, 2003.

In addition to divider elements 100 and 200, there are many other embodiments that would be within the scope of the present invention. For example, in one embodiment the dividing element could comprise a sheet of material with a strip of adhesive disposed thereon, where the sheet is rolled around one of the tip sections and adhered to itself. In another embodiment, the dividing element could comprise a piece of material, which could be wedged between adjacent tip sections such that the dividing element would remain in place until pulled out by the end user. In another embodiment, the dividing element could comprise a thin-walled tube that could be fit over the shorter of the adjacent tip sections, the lumen of the tube being fashioned to provide a friction fit. The friction fit could be achieved by the lumen of the tube either having a certain shape, which would mesh with the outer wall of the tip section on which it was to be disposed, or having an altered surface (for example, a roughened surface produced by etching, coating, etc.). In still another embodiment, a mesh-like material is stretched over one of the tip sections like a sock, thereby preventing direct contact or the covered tip section with adjacent tip section(s).

The present invention has been described above in terms of certain preferred embodiments so that an understanding of the present invention can be conveyed. However, there are many alternative arrangements for a dividing element for a split-tip catheter not specifically described herein, but with which the present invention is applicable. Although specific features have been provided, the dividing element and split-tip catheter of the present invention would equally be embodied by other configurations not specifically recited herein. The scope of the present invention should therefore not be limited by the embodiments illustrated, but rather it should be understood that the present invention has wide applicability with respect to catheter systems generally. All modifications, variations, or equivalent elements and implementations that are within the scope of the appended claims should therefore be considered within the scope of the invention.

1. A dividing element for use with a split-tip catheter, comprising a body having a proximal end and a distal end, said body comprising a first opening and a second opening spaced apart from one another by a distance sufficient to ensure that an inner surface of a first tip section is separated from an inner surface of a second tip section when said body is positioned therebetween.

2. The dividing element according to claim 1, wherein said first tip section has a length less than a length of said second tip section, and wherein said distance is greater than or equal to the length of said first tip section.

3. The dividing element according to claim 1, wherein a tension is created between said first and second openings and the respective tip section inserted therethrough.

4. The dividing element according to claim 1, wherein said distal end is configured for attachment to a packaging tray.

5. The dividing element according to claim 1, wherein a distance between said first opening and said proximal end is less than a distance between said second opening and said distal end.

6. The dividing element according to claim 1, wherein said first and second openings comprise slits.

7. The dividing element according to claim 6, wherein said slits are in the shape of an “X”.

8. The dividing element according to claim 1, wherein said body has a length in the range of approximately 1 inch to 20 inches.

9. The dividing element according to claim 1, wherein said body is comprised of polyethylene.

10. The dividing element according to claim 1, wherein said body further comprises a weakened area between said first and second openings.

11. The dividing element according to claim 1, wherein said body further comprises a weakened area between at least one of said openings and an edge of said body.

12. In combination a split-tip catheter and a dividing element, said dividing element comprising a body having a proximal end and a distal end, said body comprising a first opening and a second opening spaced apart from one another by a distance sufficient to ensure that an inner surface of a first tip section is separated from an inner surface of a second tip section when said body is positioned therebetween.
